(19) United States
(12) Plant Patent Application Publication
(10) Pub. No.: US 2003/0154532 P1
(43) Pub. Date: Aug. 14, 2003

(54) Drought tolerant plant strain of Cymbopogan Commutatus

(76) Inventors: Ashok Kumar Shahi, Jammu (IN); Satya Narayan Sharma, Jammu (IN); Swadesh Pal, Jammu (IN); Suresh Chandra, Jammu (IN); Prabhu Dutt, Jammu (IN); Surendra Singh Balyan, Jammu (IN); Maharaj Krishen Bhan, Jammu (IN); Subhash Chandra Taneja, Jammu (IN); Ravinder Kumar Raina, Jammu (IN); Vijeshwar Verma, Jammu (IN); Shazia Zahoor, Jammu (IN); Maharaj Krishen Kaul, Jammu (IN); Ghulam Nabi Qazi, Jammu (IN)

Correspondence Address: BURNS, DOANE, SWECKER & MATHIS, L.L.P., Post Office Box 1404, Alexandria, VA, 22313-1404, US

(21) Appl. No.: 10/067,808

(22) Filed: Feb. 8, 2002

(51) Int. Cl. A01H 5/00

(52) U.S. Cl. PLT/384

(57) ABSTRACT

A new and distinct plant strain of Cymbopogon designated as Cymbopogon RLJCC1 characterized by its higher yield of oil which is rich in geraniol and geranyl acetate, which is drought tolerant, having faster regeneration and vigorous growth.

### FIELD OF THE INVENTION

The present invention relates to development of “drought tolerant strain of Cymbopogon rich in geraniol and geranyl acetate designated as Cymbopogon RLJCC1”.

### BACKGROUND OF THE INVENTION

Indian sub-continent is rich in genetic diversity in aromatic plants. The aromatic grasses viz., Cymbopogon and Vetiveria zizanoides have been used by man from ancient times both in medicine and perfumery. Genus Cymbopogon belonging to family Poaceae, are rich sources of aroma chemicals especially terpenoids. These chemical compounds present in varying concentrations in the species, varieties, ecotypes or chemotypes of Cymbopogon have great scope for utilization in perfumery, flavor and pharmaceutical industry. There are up to 60 species of Cymbopogon native to tropical and sub-tropical regions of Africa and Asia. Corrigan, D 1992. In: Adverse Effects on Herbal Drugs Vol. I Springler verlag, Berlin. 115-123. Out of 27 species available in India, mainly C.flexuosus, C.winterianus and C.martinii var. motia have been exploited for commercial cultivation as a source of citral, citronellal and geraniol respectively. Cymbopogon Commutatus (Steud.) Stapf is surviving in sub-tropical environment of Jammu district near R.S.Pura Tehsil, India. The massive collection was made during monsoon season, 1994.

The occurrence of Cymbopogon Commutatus is reported from Sudan Banthorpe, D. V., Duprey, R. J. H., Hassan, M., Janes, J. F. and Modawi, B. M. 1976. Planta Medica 29:10-19.

Pakistan, Somalia, Tanzania, Iraq and Northern India. Nasir, E and Ali, S. I. 1982. Flora of Pakistan-Poaceae, No. 143. University of Karachi, Karachi, Pakistan.

India's share in land resources of the world is only 2% on which 18% of the world's population and 15% of the world's livestock survive. The geographical matrix of India based on the reported area of 305.011 million hectares is broadly grouped into three sectors—agriculture sector (59.27%), ecological sector (33.56%) and non-agricultural sector (7.17%). Prasad, R. N. and Biswas, P. P. 2000. Land resource in sustainable agricultural development—issues and strategies Indian Farming 49(11): 9-13. Dryland agriculture in India is now practiced on 100 million hectares or 70% of the total arable land of 143.8 million ha. Crop production on these lands is dependent entirely on natural precipitation. This information suggests that there is need to develop drought tolerant variety of C.commutatus.

Present annual demand of geraniol in India stands at about 100 tonnes which is likely to increase during the coming years. In India, the current production is only 50 tonnes/year. The geraniol containing oil can be used for imparting rosaceous aroma to the wide range of products from perfumery.

Polymerase Chain Reaction (PCR) technique has found a number of applications in the molecular biology. Recent advances in PCR have made this technique one of the most powerful tools for a wide spectrum of molecular analysis, such as genome mapping. Benito C., Figueiras, A. M., Zaragoza, C.,Gallego, F. J., and De la Pena, A., (1993). Plant Mol. Biol., 21:181-183., molecular evolution Brown, P. T. H., Lange, F. D., Kranz, E, and Lorz, Hhhh., 1993. Mol. Gen. Genet., 237: 311-317., gene tagging, molecular taxonomy, diagonosis of genetic diseases and forensic sciences Erlich, H. A. D. Gelfand, and J. J. Sninsky. 1991. Science 252: 1643-1651. Randomly Amplified Polymorphic DNA profiling (RAPD) is one of the PCR techniques which is an amplification-based nucleic acid scanning technique driven by synthetic oligodeoxynucleotide primers of arbitrary sequence producing characteristic DNA fingerprints capable of detecting sequence polymorphism in anonymous nucleic acid templates. In this technique the amplification of genomic DNA using random short primers results in multiple amplification products representing amplicons randomly distributed throughout a genome which can be resolved by agarose gel electrophoresis and visualized by ethidium bromide staining.

The polymorphism obtained using RAPD result from point mutations, insertions, deletions, and inversions occurring in the respective genomes in due course of time. RAPDs are usually dominant markers that 2 are inherited in simple Mendelian fashion. No references are however available on the RAPD analysis of Cymbopogons. However the methodology used by D.Godwin, N.Sangduen R.Kunanuvatchaidach, G.Piprridis, and S.W.adkins. 1996. Plant Cell Reports 16: 320-324; Taku Ohmori, Minoru Murata and Fusao Motoyoshi. 1995. Jpn. J. Genet. 70; 179-184; F. N. Wachira, R.Waugh, C. A. Hackett, and W.Powell.(1994). Genome 38: 201-210 have been used for the present studies.

A very well established method of mass selection for developing a better strain from wild collection of Cymbopogon Commutatus was adopted during 1994 and individual plant progenies were raised vegetatively by slips.

Similar looking uniform progenies having desired phenotypic characters e.g. tiller plant, high rate of tillerization, fresh herbage, essential oil content (%) and oil quality indices (ratio of geraniol/geranyl acetate to citral) were bulked for seed formation.

Seed raised progenies exhibited phenotypic variations and a single plant of desired characters was selected and designated as RLJCC1 and further multiplied vegetatively. The studies were continued from 1995 to 1999 for further evaluation of essential oil quality stability and population adaptability.

The best plants were identified based on their superiority and outstanding drought resistibility. These were again screened out to produce a new population where after further cycles of selection were carried out based on drought resistance and chemical constituents such as geraniol and geranyl acetate.

### OBJECTS OF THE INVENTION

The primary object of the present invention is to develop new Cymbopogon strain capable of growing in natural drought conditions.

Another object of the present invention is to develop a Cymbopogon strain which exhibits the presence of geraniol and geranyl acetate as major chemical constituents having low citral content for applications in perfumery and flavor industry.

Yet another object of the present invention is to develop a Cymbopogon strain useful for utilizing the marginal as well as waste land through cultivation for production of geraniol and geranyl acetate as well as ocimene and also to generate employment of peoples of different section of societies which include farming communities, tribal communities and weaker section of the society.

### SUMMARY OF THE INVENTION

This invention relates to the isolation and development of drought tolerant variety of Cymbopogon Commutatus named as RLJCC1 through mass selection technique which is potential source of geraniol and geranyl acetate of over 80% and optimal oil content (0.4-0.5%) on fresh weight basis and characterized as drought tolerant having perennial densely tufted grass and rich herbage with mass flowering pattern.

### DETAILED DESCRIPTION OF THE INVENTION

A new and distinct hybrid plant named “RLJCC1” having the following combination of characters:

### Taxonomic Characteristics

Cymbopogon is closely allied to Andropogon and Hyparrhenia and is sometimes quite difficult to separate from them. A useful diagnostic character is the aromatic flavor when a leaf of Cymbopogon is chewed, the other genera are tasteless. The genus is notorious for the considerable variations within species and the weak separation between them. Consequently its taxonomy is still in a fluid state with differing opinions about the level at which specific rank should be accorded and with many of the species based upon indefinite characters of little practical diagnostic value.

### Cymbopogon

Tall robust perennials, leaf blade linear, aromatic, ligule membranous or scarious. Inflorescence composed of paired racemes borne on a short common peduncle and enclosed by a boat shaped spatheole, these densely crowded into a leafy false panicle which is often very large and complex; raceme short, each raceme base which deflexed at maturity, the lower most pair of spikelets in each raceme homogamous and resembling the pedicelled, internodes and pedicels linear. Sessile spikelets dorsally compressed, callus4obtuse inserted in the concave, lower ligule streaked with oil glands; 2 keeled, lower florets reduced to a hyline lemmma with awn from the sinus. Pedicelled spikelets caryopsis oblong.

### Cymbopogon Commutatus (Steud.)Stapf

Perennial, culms erect 15 to 150 cm high, leaf blade flat 10-50 cm long, 1-4 mm wide, dull green narrowed at the base, filiform tip, basal sheaths persistent thinly pubescent, spatheoles narrowly lanceolate to narrowly elliptic, 2-2.6 cm long. Racemes 15-40 mm long, lower most pedicel swollen and barrel shaped intemodes and pedicells densely ciliate along the margins glabrous to minutely puberulous on a back. Sessile spikelets narrowly lanceolate 4-7 mm long, lower glume flattish to deeply concave on the back. Upper lemma deeply bifid, with an awn 10-20 mm long. Chromosome number 2n=20, 40 Nasir, E and Ali, S. I. 1982. Flora of Pakistan-Poaceae, No. 143. University of Karachi, Karachi, Pakistan.

### Selection Strategy for Isolation of Improved Drought Tolerant Strain of C.commutatus

Cymbopogon species produce numerous intermediate forms (hybrid complexes) due to cross-pollination in nature. However, the formation of essential oil is a genetically controlled phenomenon but abiotic/biotic factors influence the oil production by stimulating the physiological processes and ultimately these changes lead to adaptation and help for the selection of a drought tolerant plant variety. Hence an improved clone RLJCC1 had been isolated by mass selection technique having same oil percentage as that of the mother plant and has better drought tolerance value than that of the parent plant. Wilde, S. A., Corey, R. F., Iyer, J. G. and Voigt, G. K. 1979. In: Soil and Plant Analysis for Tree Culture Oxford & IBH Publishing Co., New Delhi.

### Morphology of Cymbopogon RLJCC1

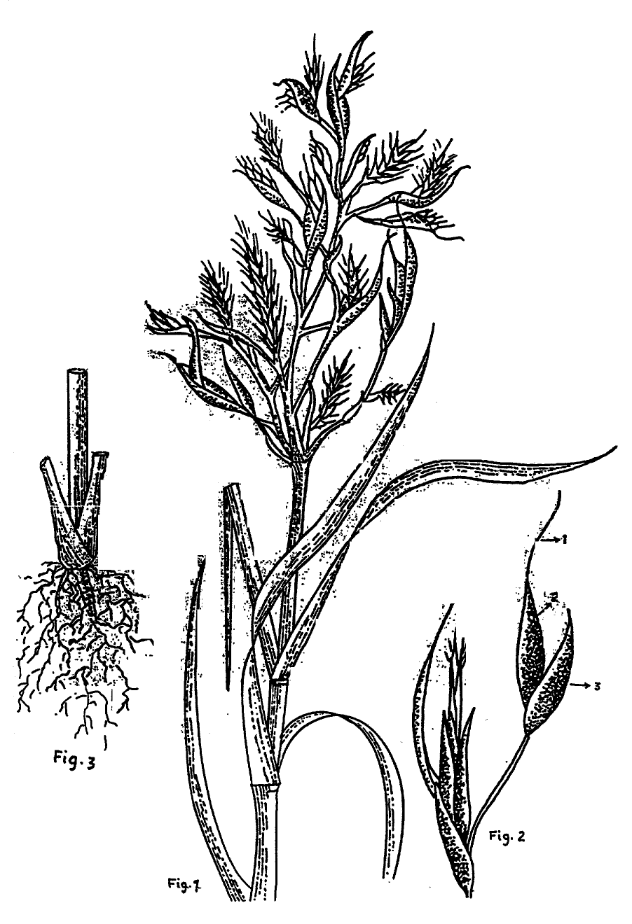

It is perennial densely tufted grass, attains a height of 100 to 150 cm having 50 cm long linear green leaf blade and 5-8 mm wide (FIGS. 1, 2 & 3). It exhibited synchronous mass flowering pattern and triggered from autumn season onwards.

### Plant Colour Description

The color description of mother plant of C.commutatus and RLJCC1 are described as per the Methum Handbook of colors by A.Komerup and J. H. Wanscher revised by Don Parey, Third Edition, 1978, published by Erye Methuen, London, having arrays of 12666 color samples. The book does not provide rough color identification but a universal ready reference for all color users. Therefore, the color description has been made as under:

### Mother Plant of C.commutatus

The color of the plant is jade green exhibiting sample reference 27E5. It refers to the color of the plant, which can be found on plate 27, Column E, Row 5.

### RLJCC1

The plant exhibited foliage green color when viewed from a distance and sample reference 30D5 i.e. plant color can be found on plate 30, Column D, Row 5.

### Morpho-Economic Characters

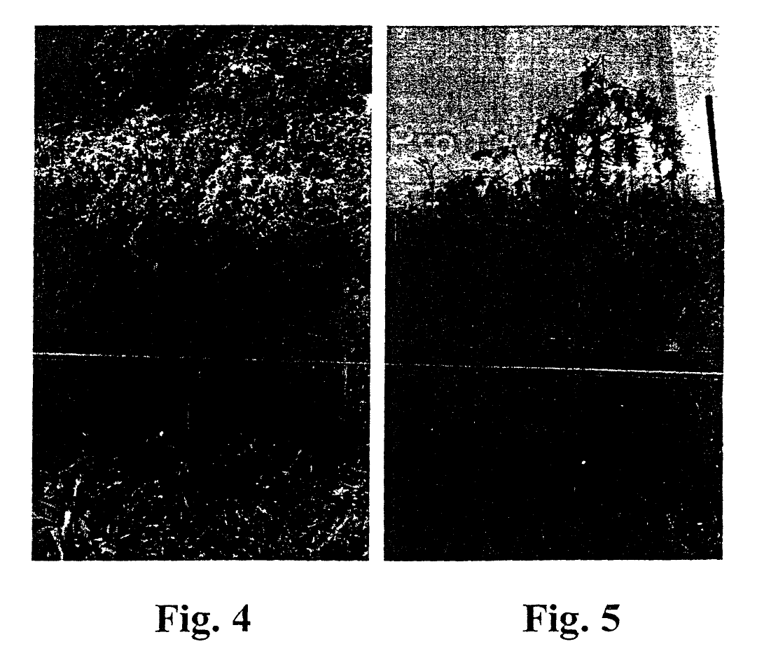

Data pertaining to morpho-economic characteristics of Cymbopogon Commutatus (RLJCC1) revealed that it exhibited better production of number of leaves and tillers plant-¹. The rate of tillerization (RTR) is as high as 2.0, which exhibited faster growth and regrowth characteristics (FIGS. 4,5,6 & 7). Essential oil yield plant-¹ is also optimal (Table 1).

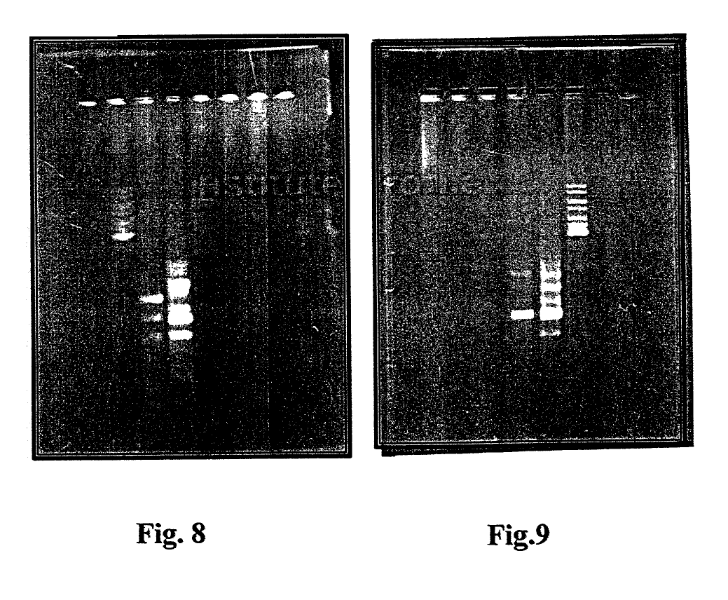

Here this RAPD Analysis has been applied to develop DNA fingerprints and relatedness of accessions of Cymbopogon species developed in this laboratory (selected plant RLJCC1, its mother plant CCM and plant similar to it chemically RRL-CN5) for quality control and identification. Protocols for the isolation of genomic DNA from hybrid plant Cymbopogon commutatus (RLJCC1), its mother plant (CCM) and its chemically comparable plant species C.nardus var. confertiflorus (RRL-CN5) were optimized by modifying the CTAB method (Fang et al., 1992). The purity of the DNA was checked spectrophotometerically at 260/280. PCR protocols for the development of comparable & reproducible RAPD profiles in selected plant species (RLJCC1, CCM and RRL-CN5) were optimized using 40 Operon Random primers showed the comparable RAPD profiles in the above Cymbopogon accessions. The best profile has been developed using the random primers 622, 27 and 29 (FIGS. 8 & 11).

### Plant Maturity and Quality Indices

Leafing and tillerization started with the rise of ambient temperature from March onwards after planting the slips in the month of February. Vegetative phase continued up to April thereafter flowering started. Floral induction continued up to 5th leafing & 6th leafing stage during May and June. Plant maturity in terms of quality indices i.e. geraniol and geranyl acetate content over 80% having optimal oil content 0.45-0.50%. Ocimene(5.35%), Linalool, (1.44%), neral(5%), geraniol(2.65%) are among the minor chemical constituents.

### Plant Stability

Comparative performance of essential oil quality index stability parameters of strain RLJCC1 and mother plant of Cymbopogon Commutatus is depicted in Table 1 which clearly indicated that strain RLJCC1 exhibited relatively better and consistent quality index stability values over the studied periods having high geraniol content, optimal range of geranyl acetate and very low citral content than those of C.commutatus mother plant. No marked differences in essential oil contents of mother plant and strain RLJCC1 were observed.

TABLE 1
Essential oil quality index values and essential oil
content of mother plant and selectant RLJCC1 (under rainfed)

| | Percentage of | | | | |
|---|---|---|---|---|---|
| Plant | | | Total of | | Essential oil |
| selectant | | | geraniol | | Content |
| Studied | | | and | | (%, w/w) on |
| Period | Geran- | Geranyl | geranyl | | fresh weight |
| (year) | iol | acetate | acetate | Citral | basis |
| Mother plant | 34.40 | 28.00 | 62.40 | 18.33 | 0.4-0.5 |
| (1994) | | | | | |
| Selectant RRLJ | 59.65 | 19.93 | 79.58 | 14.40 | 0.4-0.5 |
| CC1(1995) | | | | | |
| Selectant | 62.16 | 18.08 | 80.24 | 12.75 | 0.4-0.5 |
| (1996) | | | | | |
| Selectant | 65.90 | 16.64 | 82.54 | 10.31 | 0.45-0.5 |
| (1997) | | | | | |
| Selectant | 67.37 | 15.22 | 82.59 | 6.35 | 0.45-0.5 |
| (1998) | | | | | |
| 1999RLJCC1 | 74.50 | 9.20 | 83.70 | 5.0 | 0.45-0.5 |

### Plant Adaptability

Cymbopogon Commutatus (RLJCC1) has high survival under adverse environmental conditions due to the presence of drought tolerance value, which is as high as 12% coupled with faster regeneration and early vigorous growth. Hence, it can withstand meteorological drought. The drought tolerance values of other Cymbopogon species having similar chemical composition are 8 presented in Table 2.

TABLE 2
Drought tolerance value of Cymbopogon species

| Plant species | Drought toterance value (%) |
|---|---|
| Cymbopogon Commutatus RLJCC1 | 12 |
| Cymbopogon Commutatus | 10.5 |
| Cymbopogon nardus var. | 9 |
| confertiflorus RRL-CN-5 | |

### Variety Description Information

This invention is directed to methods for production of drought tolerant variety of C.commutatus by mass selection method under Jammu conditions and finally evaluated under field trials at sub-tropical belts/drought prone areas of District Kathua (Jammu & Kashmir State) as depicted in Table 3.

TABLE 3
Morpho-economic characters of drought
tolerant variety of C.commutatus

| | Plant species | | |
|---|---|---|---|
| | Values | | |
| Parameter(s) | RLJCCI | C.commutatus | RRL-CN-5 |
| Plant height | 104 | 109 | 115 |
| (to flowering tip)(cm) | | | |
| Number of tillers plant-1 | 66 | 30 | 40 |
| Rate of tillerization | 2.0 | 1.8 | 1.9 |
| Survival of plants (%) | 85 | 75 | 80 |
| Herbage plant (g)(Semidry) | 175 | 170 | 200 |
| Number of leaves | 159 | 152 | 150 |
| Leaf length (cm) | 50 | 48 | 60 |
| Leaf width (mm) | 68 | 6.5 | 6.5 |
| Essential oil content (% w/w) | 0.45 | 0.40 | 0.45 |
| Essential oil production | 78.8 | 75.0 | 80.0 |
| hectare-1(liters) | | | |

Other plant characteristics are as under:

A. Quality Indices

Geraniol (%)=74.5

Geranyl acetate (%)=9.2

Citral (%)=3.0

B. Plant Maturity

Best adapted for drought prone areas of the sub-tropical belts.

Requirement of growing degree days for attainment of crop maturity in terms of essential oil quality indices at blooming & 6th leafing phenophase≈2900 (° C. days)

C. Color Description

Foliage green colors and sample reference No. 30D5.

D. Odour Evaluation of Essential Oil

Grassy green note along with rosy note.

A spicy suggestion is also present.

### BRIEF DESCRIPTION OF THE ACCOMPANIED DIAGRAMS

FIG. 1 shows the inflorescence-bearing shoot.

FIG. 2 shows pedicelled & sessile spikelets (2.1 Palea, 2.2 Sessile spikelet, 2.3 Pedicellate spikelet)

FIG. 3 shows the adventitious roots

FIG. 4 shows mother plant of Cymbopogon Commutatus

FIG. 5 shows drought tolerant strain RLJCC1 (C.commutatus)

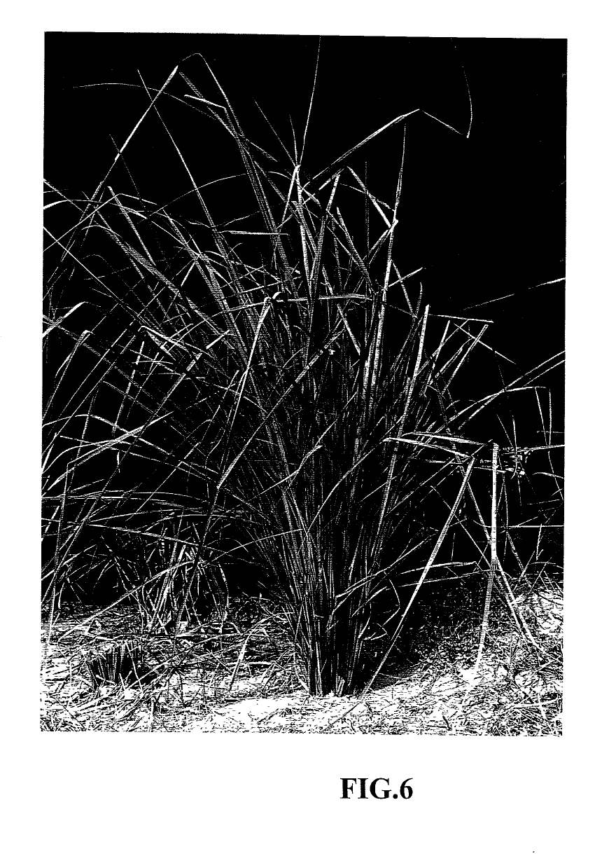

FIG. 6 shows well-grown plant of RLJCC1

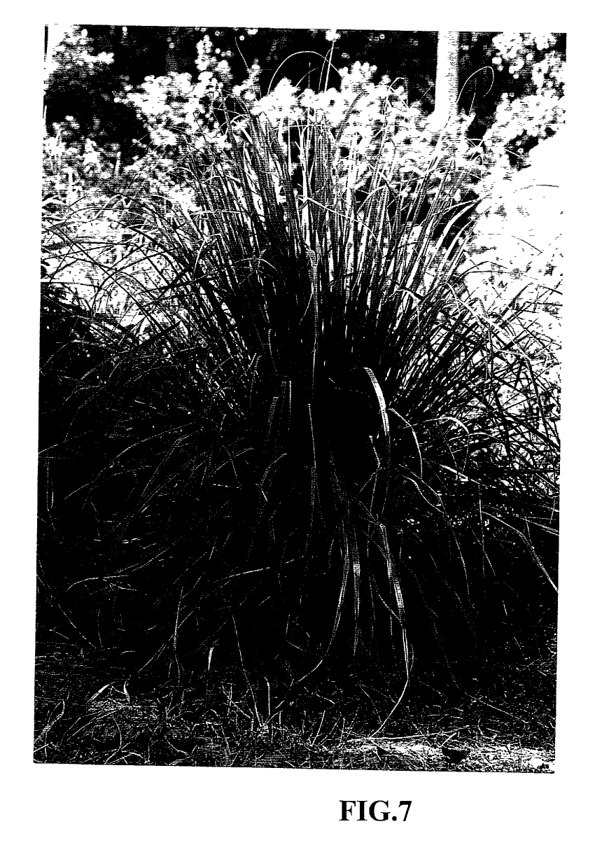

FIG. 7 shows field view of RLJCC1

FIG. 8 shows the RAPD profile of Cymbopogon Commutatus (RLJCC1) and its mother plant C.commutatus (CCM) with primer 27 using optimum concentrations: primer 27 (48 ng) and MgCl2 concentration of 2.5 mM. The RAPD profile of primer 27 in C.commutatus (RLJCC1) & its mother plant C.commutatus (CCM) is depicted in Lanes 3 & 4, respectively.

FIG. 9 shows the RAPD profile of Cymbopogon Commutatus (RLJCC1) and its mother plant C.commutatus (CCM) with primer 27 using optimum concentrations: primer 27 (48 ng) and MgCl2 concentration of 2.5 mM. The RAPD profile of primer 27 in C.commutatus (RLJCC1) & its mother plant C.commutatus (CCM) is depicted in Lanes 3 & 4, respectively.

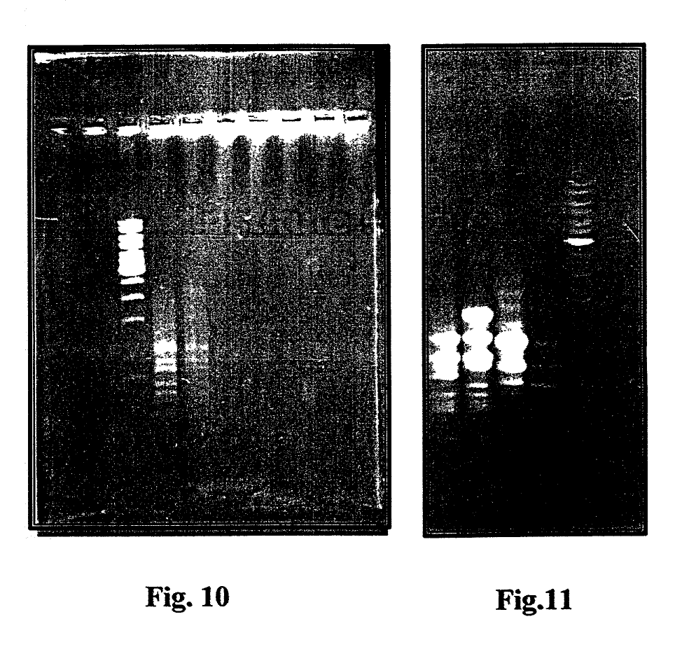

FIG. 10 shows the RAPD profile of C.commutatus (RLJCC1) and its chemically comparable plant species C.nardus var. confertiflorus RRL-CN5 with primers 27 and 29 using optimum concentrations of the primer 27 (48 ng) and MgCl2 (2.5 mM) in both the plants species. In case of primer 29, the best profile was obtained using primer concentration of 33 ng and MgCl2 concentration of 2.5 mM. The RAPD profile of primer 27 in C.commutatus (RLJCC1) & C.nardus var. confertiflorus (RRL-CN5) is depicted in Lanes 1&2 while that of primer 29 in C.commutatus (RLJCC1) and C.nardus var. confertiflorus (RRL-CN5) is depicted in Lanes 3&4, respectively.

FIG. 11 shows the RAPD profile of primer 22 in C.commutatus (RLJCC1) & its chemically comparable plant species C.nardus var. confertiflorus (RRL-CN5) using optimum primer concentration (33 ng) & MgCl2 concentration (2.5 mM). The RAPD profile of primer 22 in C.commutatus (RLJCC1) & C.nardus var. confertiflorus (RRL-CN5) is depicted in Lanes 3&4. The exact PCR protocol followed is given as under:

The RAPD profiles of the selected hybrid (RLJCC1), its mother plant (CCM) and the plant related to it chemically (RRL-CN5) using the above mentioned primers (Table 4) are very specific and can be used for the identification of the specific cultivars/chemotypes.

TABLE 4
Name and sequence of primers used

| Primer code | Operon Random primer | Primer sequence |
|---|---|---|
| 22 | OPA02 | TGCCGAGCTG |
| 27 | OPA07 | GAAACGGGTG |
| 29 | OPA09 | GGGTAACGCC |

The PCR protocol:

$\begin{matrix}92 –95° C . - & 2 –4 min \times 1 cycle \\ \begin{matrix}92 –95° C . - \\ 32 –38° C . - \\ 72 ° C . -\end{matrix} & \begin{matrix}1 –2 min \\ 1 –2 min \\ 1.5 –2 .5 min\end{matrix} \} \times 30 –45 Cycles \\ 72 ° C . - & 4 –7 min \times 1 cycle\end{matrix}$

The reaction assay mixture prepared as under:

DNA—7-20 ng

10× buffer—2-3 μl

MgCl2—1.5-2.5 mM

dNTP mix—150-250 mM

H2O—As per requirement

Primer—20-40 ng

Taq. Pol. Enz.—0.5-2.0 U 20-30 μl reaction

FIG. 12 shows mother plant of C.commutatus (CCM) at vegetative stage.

FIG. 13 shows droughts tolerant strain RLJCC1 at vegetative stage. (The FIGS. 12 & 13 are for foliage color descriptions).

### ADVANTAGES

1. High regeneration vigor of the tillers.

2. More than 85% survival of tillers and faster growth capacity and quick leaf induction. Hence, it would be an ideal variety for commercial cultivation especially under drought prone areas/belts of the Indian regions.

3. C.commutatus (RLJCC1) essential oil can be used extensively for imparting rosaceous aroma to wide range of products in perfumery, soaps & flavor and food industry. Apart from the occurrence of geraniol (74.5%) and geranyl acetate (9.20%) in its essential oil, a high value product ocimene (5-7%) is also present, which is extensively used in high-grade perfumes.

4. The essential oil is devoid of unusual menthadienols, as reported in Sudanese C.commutatus essential oil up to 87%. Banthorpe, D. V., Duprey, R. J. H., Hassan, M., Janes, J. F. and Modawi, B. M. 1976. Planta Medica 29:10-19.

5. This oil also provides protection against mosquito bite i.e., acts as a mosquito repellent.

1. A drought resistant strain of Cymbopogon code named as RLJCC1, as described and illustrated, and having the following characteristics:
(a) isolated by mass selection technique from the wild collection of Cymbopogon Commutatus,
(b) the plant characterized as drought tolerant having perennial densely tufted grass and rich herbage with mass flowering pattern.
(c) the plant rich in geraniol (74.5%) and geranyl acetate (9.2%) and
(d) the plant having a unique RAPD profile against primers OPA02, OPA07 & OPA09.(Operon Random Primer Codes).
